(19) United States
(12) Patent Application Publication
(10) Pub. No.: US 2016/0045336 A1
(43) Pub. Date: Feb. 18, 2016

(54) PROSTHETIC FEEDING DEVICE AND A MULTI-POSITION PROSTHETIC DEVICE

(71) Applicant: Amputee Mobility Products, Azle, TX (US)

(72) Inventors: James Mike Cordell, Azle, TX (US); David Worley, Azle, TX (US)

(21) Appl. No.: 14/825,940

(22) Filed: Aug. 13, 2015

Related U.S. Application Data
(60) Provisional application No. 62/036,961, filed on Aug. 13, 2014.

(51) Int. Cl. A61F 2/58

(52) U.S. Cl. CPC A61F 2/588; A61F 2/583

(57) ABSTRACT

A prosthetic feeding device and a multi-position prosthetic device are provided herein. In one embodiment, the prosthetic feeding device includes: (1) a multiple position rotator having (1A) a main body having a shaft tunnel and a rotation director, (1B) a rotating body having a pivot shaft and a head with a device receptor, wherein the rotation director is located within the main body and positioned to permit rotation of the pivot shaft within the shaft tunnel in either a clockwise or counter clockwise direction around a longitudinal axis of the main body, and (1C) a cap screw mechanically coupled to the pivot shaft to position the pivot shaft in the shaft tunnel; and (2) an eating utensil mechanically coupled to the head via the device receptor.

### CROSS RELATED APPLICATION

This application claims priority from U.S. Provisional Application No. 62/036,961 filed on Aug. 13, 2014, BY Worley et. al, entitled “ROTATING END DEVICE WITH A MULTI-POSITION ROTATING DEVICE,” which is incorporated herein by reference in its entirety.

### TECHNICAL FIELD

This application relates to prosthetics and orthotics. More particularly, the application relates to improving the independence of individuals with mobility impairments and/or upper-limb impairments.

### BACKGROUND

Conventional amputee and mobility eating utensils are swivel type devices that make it difficult for an individual to feed themselves. For example, these conventional eating utensils prevent, or at least hinder, a user from securing food from a plate with a fork or spoon and then effectively transitioning the food from the plate to their mouths without some type of secondary assistance. Devices, systems or utensils that could improve the independence of individuals, such as allowing users to feed themselves, would be welcomed in the art.

### SUMMARY

In one aspect, a prosthetic feeding device is disclosed. In one embodiment, the prosthetic feeding device includes: (1) a multiple position rotator including (1A) a main body having a shaft tunnel and a rotation director, (1B) a rotating body having a pivot shaft and a head with a device receptor, wherein the rotation director is located within the main body and positioned to permit rotation of the pivot shaft within the shaft tunnel in either a clockwise or counter clockwise direction around a longitudinal axis of the main body, and (1C) a cap screw mechanically coupled to the pivot shaft to position the pivot shaft in the shaft tunnel; and (2) an eating utensil mechanically coupled to the head via the device receptor.

In another aspect, a multi-position prosthetic device is disclosed. In one embodiment, the multi-position prosthetic device includes: (1) a main body having a shaft tunnel and a rotation director and (2) a rotating body having a pivot shaft and a head with a device receptor configured to mechanically couple an end device to the head, wherein the rotation director is located within the main body and positioned to permit rotation of the pivot shaft within the shaft tunnel in only one direction around a longitudinal axis of the main body.

In yet another aspect, the disclosure provides another embodiment of a prosthetic feeding device. In this embodiment, the prosthetic feeding device includes: (1) a multiple position rotator having: (1A) a main body having a shaft tunnel and a one way bearing device and (1B) a rotating body having a pivot shaft with a threaded coupling receiver and a head with a device receptor, wherein the one way bearing device is positioned to surround a portion of the shaft tunnel to permit and control rotation of the pivot shaft within the shaft tunnel in either a clockwise or counter clockwise direction around a longitudinal axis of the main body and (2) an eating utensil mechanically coupled to the head via the device receptor.

### BRIEF DESCRIPTION OF THE DRAWINGS

For a more complete understanding of the present disclosure, reference is now made to the following descriptions taken in conjunction with the accompanying drawings, in which:

FIG. 1 illustrates a side view of an embodiment of a multi-position prosthetic device constructed according to the principles of the disclosure;

FIG. 2 illustrates an exploded view showing parts of an embodiment of a multiple position rotator of the multi-position prosthetic device of FIG. 1;

FIG. 3A illustrates a side view and FIG. 3B illustrates an end view of another embodiment of a main body constructed according to the principles of the disclosure; and

FIG. 4 illustrates a side view of an embodiment of the rotating body of the multi-position prosthetic device of FIG. 1.

### DETAILED DESCRIPTION

The disclosure provides embodiments of a multi-position prosthetic and orthotic device, referred to herein as a multi-position prosthetic device, which can be employed by individuals to assist them in various tasks. The multi-position prosthetic device includes a multiple position rotator for one-way rotation that allows active positioning of an end device. An end device is a device configured for a specific task or tasks that is mechanically couplable to the multiple position rotator. When an end device is coupled or fixed to the multiple position rotator, a user can independently perform the various tasks. For example, the multi-position prosthetic device can be a prosthetic and orthotic feeding device, referred to herein as a prosthetic feeding device, wherein the end device is a fork or another eating utensil which can be positioned via the multiple position rotator to allow an individual to independently feed himself/herself. An end device can also be a non-eating utensil such as a razor, a comb, a brush, etc.

The end device can be mechanically coupled to the multiple position rotator via a conventional means. In some embodiments, the end device is coupled to the multiple position rotator employing a non-permanent conventional means. For example, the end device can be bolted to the multiple position rotator. A conventional type of quick connector is another example of a non-permanent type of connector that can also be used for coupling the end device to the multiple position rotator. In another embodiment, the end device can be coupled to the multiple position rotator by a permanent connection, such as via a weld.

The multi-position prosthetic device can be attached to a variety of mounting structures. As such, the multi-position prosthetic device includes an attacher that allows the application of different receiving couplings. The attacher allows the multi-position prosthetic device to interface with various mounting structures including, but not limited to, prosthetic wrist units, adaptive bracing and an impaired extremity. The attacher can be coupled to various types of receiving couplings to allow universal mounting of the multi-position prosthetic device. In one embodiment, the attacher is a threaded area for coupling to various receiving couplings of different mounting structures. In another embodiment, the attacher includes two flat areas that can be pinched by a prosthetic device, such as a Motion Control Electronic Terminal Device (ETD). An electric greifer, such as an Otto Bock Greifer, conventional hooks and myoelectric hands are additional examples of clamping devices that can be used. As such, a user can hold the multi-position prosthetic device by pinching or clamping on the attacher flat areas.

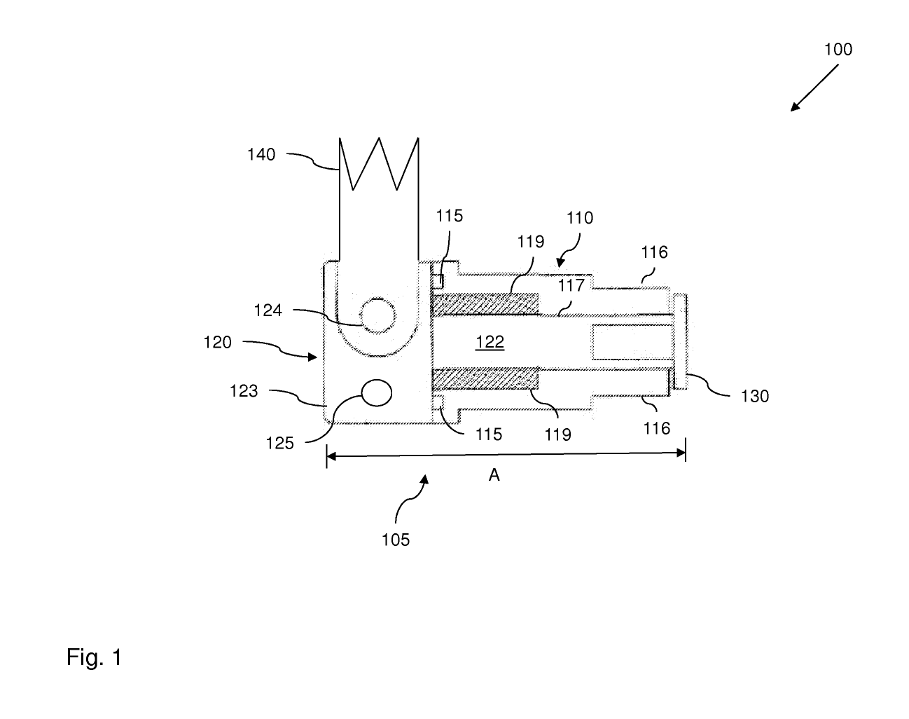

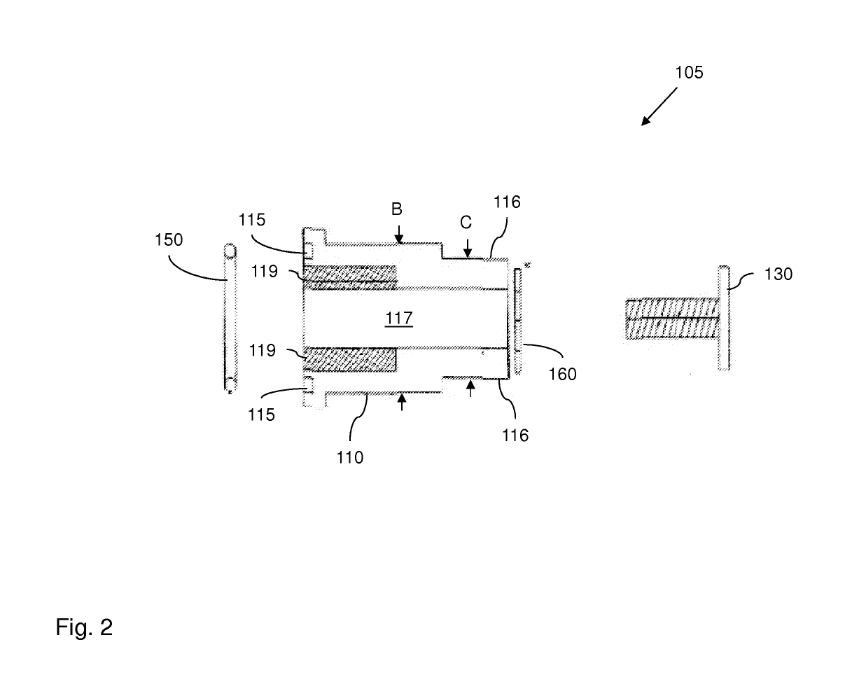

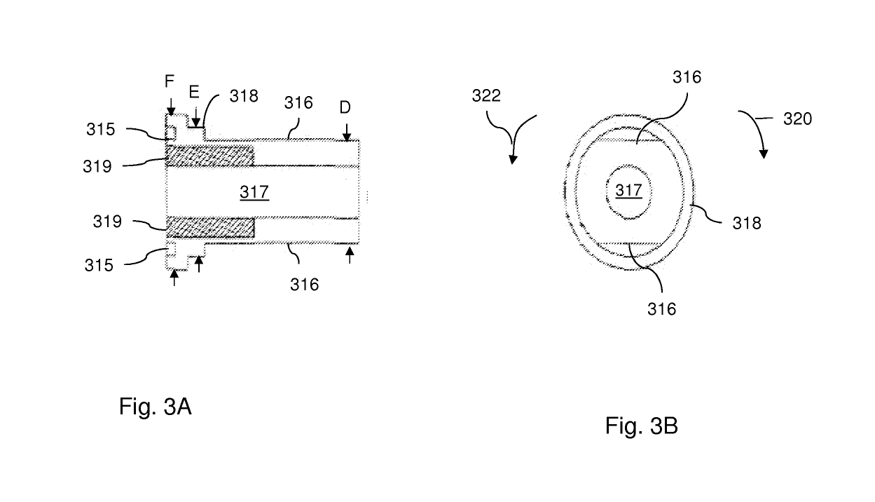

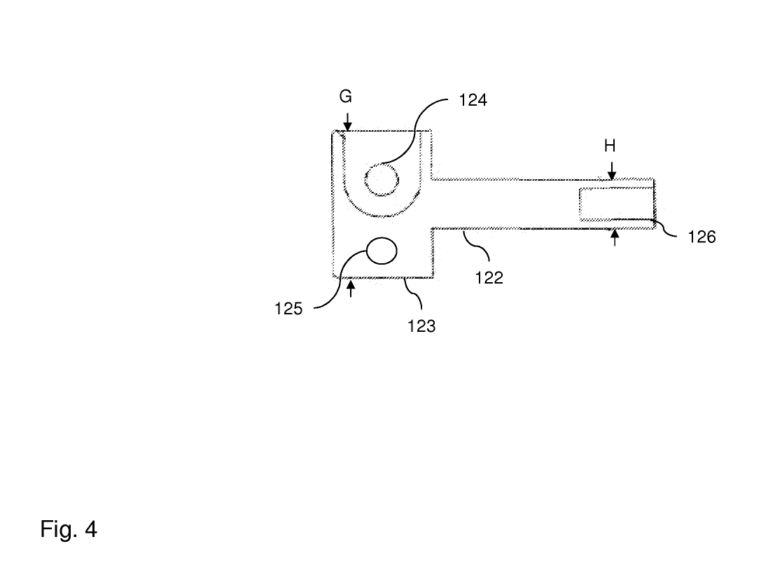

FIG. 1 illustrates a side view of an embodiment of a multi-position prosthetic device 100 constructed according to the principles of the disclosure. The multi-position prosthetic device 100 includes a multiple position rotator 105 and an end device 140. The multiple position rotator 105 includes a main body 110, a rotating body 120 and a cap screw 130. In FIG. 1, a cross section view of the main body 110 is illustrated to show the cooperation and relationship between the main body 110, the rotating body 120 and the cap screw 130 when coupled together. FIG. 2 and FIG. 3 illustrate embodiments of a main body and FIG. 4 illustrates the rotating body 120.

In one embodiment, the multi-position prosthetic device 100 or at least a portion thereof is made of steel. In another embodiment, some or the entire multi-position prosthetic device 100 is constructed of aluminum. One skilled in the art will understand that the multi-position prosthetic device 100 can be constructed of other rigid materials, also, including non-metals. In some embodiments, the multi-position prosthetic device 100, or at least a part thereof, can be constructed of a plastic or another type of moldable material. The finish of the multi-position prosthetic device 100 may also vary. For example, the finish for a metallic multi-position rotating device can be polished, anodized, or natural. Different components of the multi-position prosthetic device 100 can be constructed of different materials.

The main body 110 includes a seal groove 115, an attacher 116, a shaft tunnel 117 and a rotation director 119. The rotating body 120 includes a pivot shaft 122, a head 123, a device receptor 124, and a device receptor 125. The pivot shaft 122 fits within the shaft tunnel 117 of the main body 110 and is secured therein by the cap screw 130. The cap screw 130 fits within a coupling receiver (not illustrated in FIG. 1 but denoted as the coupling receiver 126 in FIG. 4) of the pivot shaft 122 to rotationally couple the rotating body 120 to the main body 110. A washer 160, not illustrated in FIG. 1 but shown in FIG. 2, is positioned between the cap screw 130 and an end face of the main body 110. An inner face of the head 123 is positioned against a front face of the main body 110. An o-ring 150, not illustrated in FIG. 1 but shown in FIG. 2, is positioned in the seal groove 115 in an assembled multi-position prosthetic device 100. The o-ring 150 assists with interfacing between the rotating body 120 and the main body 110 and provides friction against the inner face of the head 123 when rotating. A screw or screws, such as a set screw, can also be used to provide friction against the inner face to assist in holding a stable position. In one embodiment, the screw can protrude through a shaft in the lip area at the top or bottom of the main body 110 between the seal groove 115 and the outer edge of the main body 110. The head of the screw would be located towards the cap screw 130 to allow adjustments. The screw can be a nylon screw. The o-ring 150 can also provide a seal to keep lubricating fluid within the multi-position prosthetic device 100 and protection against outside contamination such as dirt. A lubricating fluid, such as an industrial grade lubricant, may be employed to assist in the rotation movement. In some embodiments, a lubricating fluid is not used. The lubricating fluid is located in the space between the pivot shaft 122 and the main body 110 and between the inner face of the rotational body 120 and the front face of the main body 110. The space between the pivot shaft 122 and the main body 110 in the shaft tunnel 117 is sufficient to allow rotation under the control of the rotation director 119 (i.e., rotation without the pivot shaft 122 and the main body 110 touching within the shaft tunnel 117 except where the rotation director 119 is located).

The multi-position prosthetic device 100 allows rotation of the end device 140 in a single direction along a rotational line and holds the end device 140 at stable positions along the rotational line. The rotational line can correspond to a geometric shape of the rotation director 119. The rotational line can be an arc. In one embodiment disclosed herein the arc is a circle providing 360 degrees of stable positions. The rotational line is around the longitudinal axis of the multi-position prosthetic device 100.

The stable positions can be determined by position holders of the rotation director 119. The position holders provide the stable positions along the rotation arc that allow the end device 140 to be temporarily fixed. In some embodiments, each of the stable positions is equally spaced (including substantially equally spaced) apart along the rotational line. In other embodiments, the stable positions along the rotational line are not equally spaced. As such, the positioned holders or the stable positions that correspond thereto are irregularly spaced along the rotational line. The spacing of the position holders along the rotational line may be based on, for example, expected use, user size or user preferences.

Instead of a conventional swivel device, the multi-position prosthetic device 100 allows the end device 140 connected thereto to be fixed at a stable position along the rotational line of the rotation director 119. A stable position is wherein the rotating body 120 is temporarily fixed from rotating with respect to the main body 110. A non-stable position can be when the rotating body 120 is in between the spaces defined by the position holders. A sufficient rotational force is needed to move the rotational body 120 from a stable position. Thus, when the end device 140 is an eating utensil, users can feed themselves by placing the connected eating utensil at a particular position of their choosing from the multiple stable positions provided by the multi-position prosthetic device 100. To rotate to another stable position, the user can apply a sufficient rotational force to the rotating body 120 (via the end device 140 on the edge of a table, for example). A sufficient rotational force is the amount of rotational force needed to move the rotational body 120 from a stable position. In one embodiment, the rotation director 119 only permits clockwise rotation. In another embodiment, the rotation director 119 only permits counter clockwise rotation. The rotation director 119 is housed within the main body 110 and positioned with the shaft tunnel 117 to permit rotation of the pivot shaft 122, and therefore the rotating body 120, in only one direction. In FIG. 1, the rotation director 119 is positioned to surround a portion of the shaft tunnel 117 to control the rotation of the pivot shaft 122.

In FIG. 1, the rotation director 119 is a one way bearing device, such as a one way clutch bearing, that allows rotation of the rotating body 120 in a single direction. Accordingly, depending on the type of bearings, the one way bearing allows rotation either clockwise or counter clockwise. In this embodiment, the bearings of the one way bearing device are the position holders. The one way bearing device can be a conventional device, such as the noted one way clutch bearing, that provides 360 degrees of position holders and corresponding stable positions. With a one way clutch bearing, a stable position can be the multiple locations along the 360 degrees in which a user stops the rotation.

In some embodiments, each of the stable positions is equally spaced (including substantially equally spaced) apart along the rotational line with respect to the bearings. In other embodiments, the stable positions along the rotational line may not be equally spaced. In these embodiments, spacers may be employed between the bearings of a one way clutch. The spacing of the position holders along the rotational line may be based on, for example, expected use, user size or user preferences.

The end device 140 is fixed to a portion of the head 123. In FIG. 1, a device receptor 124 receives the end device 140 which is illustrated as a fork. One skilled in the art will understand that another type of eating utensil, utensil or tool could be connected to the device receptor 124 as an end device 140 instead of a fork. Additionally, one skilled in the art will understand that a connecting component used to couple the end device 140 to the head 123 can also be coupled to the device receptor 124. In one embodiment, the device receptor 124 is a threaded hole or threaded female connector configured to receive a bolt used to couple the end device 140 to the head 123. The device receptor 124 could also be a threaded bolt or male connector. As noted above, an end device can be attached using other means, also. The location of attachment can also vary in different embodiments. For example, an end device can also be connected along the flat, outside surface of the head 123. In one embodiment, more than one attachment area can be included. Another attachment area, a device receptor 125, is denoted by the circle on the outside surface 125 of the head 123 in FIG. 1. Thus, the head 123 can include multiple device receptors as denoted by the device receptors 124 and 125. The type of attachments for end devices may be the same. In some embodiments, the various device receptors, such as device receptors 124, 125, are of different types. For example, a bolted connection can be used with a welded connection. In other embodiments, the device receptors 124, 125, can be of the same type. Each of the device receptors 124, 125, can be a conventional male or female connector. In some embodiments, the multi-position prosthetic device 100 could include two end devices spaced apart at different device receptors to allow a user to employ two different devices. For example, a user could use a fork and a spoon that are connected to the head 123 at the same time.

FIG. 1 and the other figures denote dimensions of the multi-position prosthetic device 100. Values for dimensions in FIG. 1 and the corresponding Figures of the disclosure represent one embodiment of the multi-position prosthetic device 100. Dimension “A” represents the overall length of the multi-position prosthetic device 100 along the longitudinal axis thereof. In one embodiment, “A” is 1.695 inches. One skilled in the art will understand that the dimensions can vary depending on application. The multi-position prosthetic device 100 can also be sized to correspond to the various mounting structures and end devices in which it will interact. In some embodiments, the size of the end devices can vary depending on the particular user. For example, the end device 140 (the fork) can be smaller for use by a child.

FIG. 2 illustrates an exploded view showing parts of an embodiment of the multiple position rotator 105 of the multi-position prosthetic device of FIG. 1. FIG. 2 illustrates the main body 110, the cap screw 130, the o-ring 150 and the washer 160. Diameters of different portions of the main body 110 are denoted in FIG. 2 as “B” and “C.” In one embodiment, “B” is 0.625 inches and “C” is 0.5 inches. In one embodiment the cap screw 130 is a truss screw with 8-32 threads. The main body 110 is again presented in a cross sectional view to illustrate the seal groove 115, the shaft tunnel 117 and the position director 119.

The seal groove 115 is sized and positioned to receive and hold the o-ring 150. The seal groove 115 is a machined groove configured for an o-ring. In one embodiment, the o-ring 150 is a ½×⅝ inch diameter o-ring. One skilled in the art will understand that other types of gaskets can be used instead of an o-ring and that the seal groove 115 can be configured for different sizes of gaskets or o-rings. The washer 160 can be a crush washer sized to fit around the cap screw 130 and against the main body 110. In some embodiments, an o-ring can be used instead of a washer. In one embodiment, a ¼″ by ⅜″ o-ring can be used instead of the washer 160. Conventional gaskets or o-rings can be employed in the illustrated embodiment.

The main body 110 is designed to be used with mobility units that are standard within the orthotic and prosthetic industry. The attacher 116 is configured to provide a mechanical connection to other devices, such as prosthetic limbs, for the multi-position prosthetic device 100. In the illustrated embodiment, the attacher 116 is a threaded area to provide a male attachment for a female receptacle of another device. The threaded area is threaded for attaching with about a ½ inch area of threads along the length of the attacher 116. In the illustrated embodiment, the attacher 116 has ½-20 threads for attaching to conventional prosthetic limbs in the U.S. The diameter of the attacher 116 and the thread type can vary in different embodiments to correspond to various standard coupling devices. For example, the diameter and thread type may be configured to correspond to European prosthetic limb standards. Thus, the attacher 116 can be sized and shaped to comply with various attachment standards throughout the world.

In other embodiments, the attacher 116 can be configured to couple with devices employing a quick-disconnect connection or other conventional coupling mechanisms. One skilled in the art will understand that a quick-disconnect connection includes a sleeve with ball bearings that allows the attacher 116 to snap into place. For this type of connection, the outer diameter of the attacher 116, or dimension “C,” is ⅝ inch. The attributes of the attacher 116, including the diameter, length and shape, can vary to connect to designated receivers. The configuration of the attacher 116 can also vary. For example, the attacher 116 is a male device that couples with a type of female device. In some embodiments, at least a portion of the attacher 116 may be a female device that is configured to couple to a male receiving device. In one embodiment, the attacher 116 is configured to connect to multiple types of connections. For example, the attacher 116 can include a threaded area on the outside surface and also configured with a disconnect connection. Thus, a single embodiment can be configured for multiple types of connections.

FIG. 3A illustrates a side view and FIG. 3B illustrates an end view of another embodiment of a main body, main body 318, constructed according to the principles of the disclosure. The main body 318 is designed to be used with amputee patients. The main body 318 functions as the main body 110 functions but includes an attacher 316 that differs from the threaded attacher 116. The attacher 316 includes a top and bottom flat area that allows the main body 318 to be held by a pinching or clamping device. The pinching or clamping device can be a conventional coupling device used in the industry. Dimensions “D,” “E” and “F” are denoted in FIG. 3A. In one embodiment, is 0.5 inches, “E” is 0.625 inches and “F” is 0.750 inches. Dimension “F” can also apply to the main body 110 of FIG. 1. The main body 318 also includes a seal groove 315, a shaft tunnel 317 and a rotation director 319. Each of these components can be the same as the seal groove 115, the shaft tunnel 117 and the rotation director 119 of FIG. 13.

FIG. 3B illustrates an end view of the main body 318 that shows the top and bottom flat areas of the attacher 316. FIG. 3B also indicates a clockwise rotational line 320 and a counter clockwise rotational line 322 around the longitudinal axis of the main body 318. These rotational lines represent the same clockwise and counter clockwise rotation around the longitudinal axis of the main body 110 of FIG. 1.

FIG. 4 illustrates a side view of an embodiment of the rotating body 120 of the multi-position prosthetic device of FIG. 1. The rotating body 120 includes coupling receiver 126. In one embodiment, the coupling receiver 126 is a female receiver having 8-32 threads for receiving and providing a mechanical connection for the cap screw 130. In one embodiment, dimension “G” is 0.750 inches and dimension “H” is 0.250 inches.

Although the disclosure has been described in detail, those skilled in the art should understand that they can make various changes, substitutions and alterations herein without departing from the spirit and scope of the invention in its broadest form. One skilled in the art will understand that the dimensions disclosed herein can be modified for different embodiments. Additionally, one skilled in the art will understand the scope of the disclosure includes manufacturing the various components disclosed herein and methods of manufacturing the components. In one embodiment, the various components may be machined or molded.

The disclosure provides a multi-position prosthetic device that increases the independence and quality of life of amputees or physically impaired individuals. The multi-position prosthetic device can be a prosthetic feeding device that includes at least one eating utensil connected as an end device. With the prosthetic feeding device, an individual can feed themselves by rotating an attached fork, spoon, knife, or other eating utensil to the proper orientation as needed for them. In some embodiments, the disclosed multi-position prosthetic device can be employed with additional types of position holders, such as detent screws or set screws, which can be used with a one way clutch bearing to provide or hold stable positions. For example, an additional position holder can be used to provide additional friction on the inner surface of head 123 to assist in holding a stable position.

What is claimed is:

1. A prosthetic feeding device, comprising:
a multiple position rotator including:
a main body having a shaft tunnel and a rotation director,
a rotating body having a pivot shaft and a head with a device receptor, wherein said rotation director is located within said main body and positioned to permit rotation of said pivot shaft within said shaft tunnel in either a clockwise or counter clockwise direction around a longitudinal axis of said main body, and
a cap screw mechanically coupled to said pivot shaft to position said pivot shaft in said shaft tunnel; and
an eating utensil mechanically coupled to said head via said device receptor.

2. The prosthetic feeding device as recited in claim 1 wherein said rotation director is positioned with said shaft tunnel to permit said rotation of said pivot shaft.

3. The prosthetic feeding device as recited in claim 1 wherein said main body further includes an attacher.

4. The prosthetic feeding device as recited in claim 3 wherein said attacher is a threaded male connector.

5. The prosthetic feeding device as recited in claim 1 wherein said device receptor is a threaded female receptor.

6. The prosthetic feeding device as recited in claim 1 wherein said pivot shaft includes a coupling receiver, wherein said cap screw is mechanically affixed to said pivot shaft via said coupling receiver to rotationally couple said rotating body to said main body.

7. The prosthetic feeding device as recited in claim 1 wherein said rotation director is a one-way bearing device.

8. The prosthetic feeding device as recited in claim 1 wherein said rotation director is configured to hold said rotating body at stable positions along a rotational line around said longitudinal axis.

9. The prosthetic feeding device as recited in claim 1 wherein said rotation director is positioned to surround a portion of said shaft tunnel to control said rotation of said pivot shaft.

10. A multi-position prosthetic device, comprising:
a main body having a shaft tunnel and a rotation director; and
a rotating body having a pivot shaft and a head with a device receptor configured to mechanically couple an end device to said head, wherein said rotation director is located within said main body and positioned to permit rotation of said pivot shaft within said shaft tunnel in only one direction around a longitudinal axis of said main body.

11. The multi-position prosthetic device as recited in claim 10 wherein said rotation director is positioned to surround a portion of said shaft tunnel to control said rotation of said pivot shaft.

12. The multi-position prosthetic device as recited in claim 10 wherein said main body further includes an attacher configured to connect said multi-position prosthetic device to at least one type of mounting structure.

13. The multi-position prosthetic device as recited in claim 10 further comprising a cap screw mechanically coupled to said pivot shaft to position said pivot shaft in said shaft tunnel to rotationally couple said rotating body to said main body.

14. The multi-position prosthetic device as recited in claim 10 wherein said device receptor is a threaded male connector.

15. The multi-position prosthetic device as recited in claim 10 wherein said pivot shaft is mechanically affixed to said cap screw via a threaded coupling receiver of said pivot shaft.

16. The multi-position prosthetic device as recited in claim 10 wherein said rotation director is a one-way clutch bearing having bearings that hold said rotating body in stable positions.

17. The multi-position prosthetic device as recited in claim 16 wherein said bearings hold said rotating body at stable positions along a rotational line around said longitudinal axis.

18. The multi-position prosthetic device as recited in claim 10 further comprising an end device mechanically fixed to said rotating body via said device receptor.

19. A prosthetic feeding device, comprising:
a multiple position rotator including:
a main body having a shaft tunnel and a one way bearing device, and
a rotating body having a pivot shaft with a threaded coupling receiver and a head with a device receptor, wherein said one way bearing device is positioned to surround a portion of said shaft tunnel to permit and control rotation of said pivot shaft within said shaft tunnel in either a clockwise or counter clockwise direction around a longitudinal axis of said main body; and
an eating utensil mechanically coupled to said head via said device receptor.

20. The prosthetic feeding device as recited in claim 19 wherein said multiple position rotator further includes a cap screw mechanically coupled to said pivot shaft via said threaded coupling receiver to position said pivot shaft in said shaft tunnel.
